(19) United States
(12) Patent Application Publication
(10) Pub. No.: US 2017/0056201 A1
(43) Pub. Date: Mar. 2, 2017

(54) INTERVERTEBRAL FUSION APPARATUS FOR POSITIONING AND COMBINING VERTEBRAE

(71) Applicant: WILTROM CO., LTD., HSINCHU COUNTY (TW)

(72) Inventors: HUANG-CHIEN LIANG, HSINCHU CITY (TW); YI-CHUN SU, TAOYUAN CITY (TW); HUNG-YIN TAI, HSINCHU CITY (TW); HUANG-CHI CHEN, HSINCHU COUNTY (TW)

(21) Appl. No.: 15/245,421

(22) Filed: Aug. 24, 2016

Related U.S. Application Data
(60) Provisional application No. 62/210,048, filed on Aug. 26, 2015.

(51) Int. Cl. A61F 2/44; A61B 17/88; A61F 2/46

(52) U.S. Cl. CPC A61F 2/4455; A61F 2/447; A61F 2/4611; A61B 17/8841; A61F 2002/4475; A61F 2210/0071; A61F 2210/0085; A61F 2230/0082

(57) ABSTRACT

The present invention provides an intervertebral fusion apparatus for positioning and combining vertebrae, which use a solidifiable material and an intervertebral fusion cage concurrently. By diffusing the solidifiable material from the top or bottom surface of the intervertebral fusion cage, the contact area with vertebrae and hence the stability can be increased. In addition, the intervertebral fusion cage provided by the present invention can further include a wing member connected pivotally on the side and operated concurrently with the solidifiable material to change the shape and thus avoid slipping off the operation opening. By using the intervertebral fusion apparatus, the stability of the intervertebral fusion cage during and after surgeries can be improved effectively. Thereby, the success rate and safety of vertebral fusion surgeries can be increased.

### FIELD OF THE INVENTION

The present invention relates generally to a spine surgery apparatus, and particularly to an intervertebral fusion apparatus for positioning and combining vertebrae.

### BACKGROUND OF THE INVENTION

As people age, degeneration of hard and soft tissues inside the body is unavoidable. Ageing of different parts in the body results in significantly different influences on daily living. In particular, the low back pain induced by the ageing of intervertebral discs has become one of the most annoying symptoms.

Intervertebral discs are connective tissues located between two vertebrae and act as buffers therebetween for easing the tension and pressure generated while vertebrae move and thus avoiding damages and pains on the spine due to excessively strong impacts. An intervertebral disc is normally formed by two parts. The central part is called the nucleus, which mainly provides the shock absorption capability for the intervertebral disc. The ligament attached to the adjacent vertebra and surrounding the nucleus is called the annulus, When the nucleus is dehydrated caused by losing the ability of absorbing water, it loses the shock absorption capability. Then, the pressure generated by human activities will act directly on the bone structure of the spine. By destroying the structure of the annulus, the support and structure of the whole intervertebral disc will be weakened, finally leading to direct compression on vertebrae and generating mechanical low back pains. Even worse, ageing of intervertebral discs might narrow the neural foramens surrounding the intervertebral discs or loosen the ligaments surrounding the spinal canal. Thereby, the nerve roots will be compressed and resulting in serious neuropathic pains. Both will make patients uncomfortable.

Currently, the surgery for treating the serious degenerative disc disease is mainly the spinal fusion surgery. According to the surgery, the intervertebral discectomy is first adopted to take out the degenerated intervertebral disc. Next, a graft is placed into the space between the two vertebrae after the intervertebral discectomy. Finally, pedicle screw fixators are used for fixing and fusing the vertebrae. The adopted graft can be an allograft donated by others or an autologous graft taken from the patient's own body. The implantation methods include implanting the whole bone by wedging. Alternatively, the autologous graft can be placed in a fusion cage. Then the fusion cage, which acts as a carrier, is placed into the body. Thereby, the purpose of stimulating fusion of the two vertebrae can be achieved.

The intervertebral fusion surgery using intervertebral fusion cage has become the mainstream surgery for treating intervertebral disc related diseases. The reason is because the weight distribution of vertebrae can be re-balanced, thanks to the immediate recovery of the height of intervertebral discs and of the normal curve of vertebrae. Furthermore, after the angles of vertebrae are recovered, the compression on the neural foramens surrounding intervertebral discs can be reduced, which eases immediately the neuropathic pains caused by nerve compression. In addition, the intervertebral fusion fixator also provides a highly stable environment for fusing two vertebrae with low surgical risk. Given the advantages in efficacy and safety, it naturally becomes the primary choice for treating intervertebral disc related diseases.

Unfortunately, current intervertebral fusion cages still have sonic drawbacks. First, the contact area between the implanted intervertebral fusion cage and the vertebrae is quite small, which brings about shift in the location of the intervertebral fusion cage owing to unstable friction source during the operation. Although the top and bottom surfaces of some current intervertebral fusion cages are designed zigzag for increasing the friction between the intervertebral fusion cage and the vertebrae, the improvement is still limited. In addition, because the intervertebral fusion cage is implanted into the body through the opening at the intervertebral disc, the height of the intervertebral fusion cage and the size of the opening must be pretty close. Thereby, after the operation for implanting the intervertebral fusion cage is completed, without sufficient fixation, there are some risks that the intervertebral fusion cage will slip off the operation opening for the intervertebral disc operation. For the slight consequence, it influences the efficiency of autologous recovery of vertebrae. For the severe one, nerves might be compressed and resulting in unexpected harms.

Accordingly, to provide an intervertebral fusion cage with high operative stability and free from slipping off the operation opening for guaranteeing superior surgical quality for intervertebral fusion surgeries has become the major emphasis of the field.

### SUMMARY

An objective of the present invention is to provide an intervertebral fusion apparatus for positioning and combining vertebrae. By operating the intervertebral fusion cage and a solidifiable material concurrently, the solidifiable material can diffuse to the top and bottom surfaces of the intervertebral fusion cage from the accommodating space. Thereby, the contact areas between the intervertebral fusion cage and the top and bottom vertebrae can be increased and thus improving the stability of the intervertebral fusion cage during the surgery process.

Another objective of the present invention is to provide an intervertebral fusion apparatus for positioning and combining vertebrae. By connecting pivotally a wing member to the side of the intervertebral fusion cage and extending the wing member after being implanted into the intervertebral disc location, the intervertebral fusion cage will not slip off the intervertebral disc location after completion of the surgery.

A further objective of the present invention is to provide an intervertebral fusion apparatus for positioning and combining vertebrae. By using the form change provided by the extended wing member and the contacting force provided by the solidifiable material, the intervertebral fusion cage can be combined between two vertebrae more securely.

In order to achieve the above objectives, the present invention discloses an intervertebral fusion apparatus for positioning and combining vertebrae placed between a top vertebra and a bottom vertebra. The structure of the intervertebral fusion apparatus comprises an intervertebral fusion cage including an inlet on the front surface and an accommodating space therein. The inlet communicates with the accommodating space. In addition, the intervertebral fusion apparatus further comprises a solidifiable material injected front he inlet of the intervertebral fusion cage, and entering and filling the accommodating space.

According to an embodiment of the present invention, the intervertebral fusion cage comprises a top penetrating hole and a bottom penetrating hole in at least the vertical direction. The top and bottom holes communicate with the accommodating space. Besides, the top and bottom holes include a top accommodating space and a bottom accommodating space, respectively. The solidifiable material passes through the accommodating space, and enters and fills the top or bottom accommodating space. The solidifiable material entering and filling the top or bottom accommodating space will contact the top or bottom vertebra.

According to an embodiment, the intervertebral fusion cage includes one or more side penetrating hole. A wing member pivots on the intervertebral fusion cage and located in the side penetrating hole.

According to an embodiment of the present invention, the wing member is pushed by the solidifiable material injected into the accommodating space to rotate pivotally and thus extended to the side.

According to an embodiment of the present invention, the wing member pivots on the front edge of the side penetrating hole correspondingly.

According to an embodiment of the present invention, the solidifiable material is selected from the group consisting of amorphous calcium phosphate, poorly crystalline calcium phosphate, hydroxyapatite, carbonated apatite (calcium-deficient hydroxyapatite), monocalcium phosphate, calcium metaphosphate, heptacalcium phosphate, dicalcium phosphate dihydrate, tetracalcium phosphate, octacalcium phosphate, calcium pyrophosphate, tricalcium phosphate, calcium sulfate hemihydrate, calcium sulfate dihydrate, polysaccharides, nucleic acids, carbohydrates, proteins, polypeptides, poly(α-hydroxy acids), poly(lactones), poly(amino acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide and copolymers thereof, alginic acid, arabic gum, guar gum, xantham gum, gelatin, chitin, chitosan, chitosan acetate, chitosan lactate, chondroitin sulfate, N,O-carboxymethyl chitosan, dextran (e.g., α-cyclodextrin, β-cyclodextrin, γ-cyclodextrin, or sodium dextran sulfate), fibrin glue, glycerol, hyaluronic acid, sodium hyaluronate, cellulose (e.g., methylcellulose, carboxy methylcellulose, hydroxypropyl methylcellulose, or hydroxyethyl cellulose), glucosamine, proteoglycan, starch (e.g., hydroxyethyl starch or starch soluble), lactic acid, pluronic, sodium glycerophosphate, collagen, glycogen, keratin, silk fibroin and the combination thereof.

According to an embodiment of the present invention, the solidifiable material is a thermoplastic fluid.

According to an embodiment of the present invention, the inlet of the intervertebral fusion cage includes a connecting device used for connecting with an injector.

Moreover, in order to achieve the above objectives, the present invention also discloses an intervertebral fusion apparatus for positioning and combining vertebrae comprising an intervertebral fusion cage, which includes an inlet on the front surface and an accommodating space therein. The inlet communicates with the accommodating space. In addition, the intervertebral fusion cage includes a side penetrating hole in at least the lateral direction. The side penetrating hole communicates with the accommodating space. Besides, the intervertebral fusion apparatus comprises one or more wing member pivoting on the intervertebral fusion cage and located in the side penetrating hole, and thus enabling the wing member to have contacting and connecting effects by extending outwards.

According to an embodiment of the present invention, the accommodating space further includes a solidifiable material filling to the top or bottom accommodating space.

According to an embodiment of the present invention, the wing member is pushed by the solidifiable material injected into the accommodating space to rotate pivotally and thus extended to the side.

### BRIEF DESCRIPTION OF THE DRAWINGS

FIGS. 1A and 1B show a top view and a right-side view of the intervertebral fusion apparatus for positioning and combining vertebrae in the operational state according to a preferred embodiment of the present invention;

FIGS. 2A and 2B show side views of the top and bottom surfaces of the intervertebral fusion cage of the intervertebral fusion apparatus according to a preferred embodiment of the present invention;

FIG. 3 shows a side view of the intervertebral fusion cage combined with the operational device according to a preferred embodiment of the present invention;

FIG. 4 shows a side view of the intervertebral fusion cage according to another preferred embodiment of the present invention;

FIG. 5 shows aside view of the intervertebral fusion cage according to another preferred embodiment of the present invention; and

FIG. 6 shows a top view of the intervertebral fusion apparatus for positioning and combining vertebrae in the operational state according to another preferred embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures.

According to the present invention, a novel intervertebral fusion apparatus for positioning and combining vertebrae and the method thereof are provided for solving the problems of shift of the intervertebral fusion cage from the surgical site due to unstable friction source and the intervertebral fusion cage slipping off the operation opening easily after completion of the surgery. The intervertebral fusion apparatus for positioning and combining vertebrae adopts an intervertebral fusion cage and a filling material to operate concurrently. The intervertebral fusion cage includes a central accommodating space communicating with a top opening and a bottom opening. After the intervertebral fusion cage is set up in a target positioning region during the spine surgery process, the filling material is delivered to the target positioning region from the accommodating space and the top and bottom openings of the intervertebral fusion cage for increasing the junction area with the surrounding environment. By using the limiting property of the filling material, the overall stability of the intervertebral fusion apparatus can be improved. Thereby, the problem of shift of the general intervertebral fusion apparatus from the surgical site due to unstable friction source can be overcome. In addition, the intervertebral fusion cage according to the present invention further comprises one or more side opening pivoted by a wing member. As the tilling material is pushed outwards from the one or more side opening, the wing member can be extended, and hence increasing the area of the overall intervertebral fusion apparatus for overcoming the problem of the intervertebral fusion cage slipping off the operation opening easily after completion of the surgery.

In the following, the components, properties, and disposal of the intervertebral fusion apparatus for positioning and combining vertebrae according to the present invention will be described.

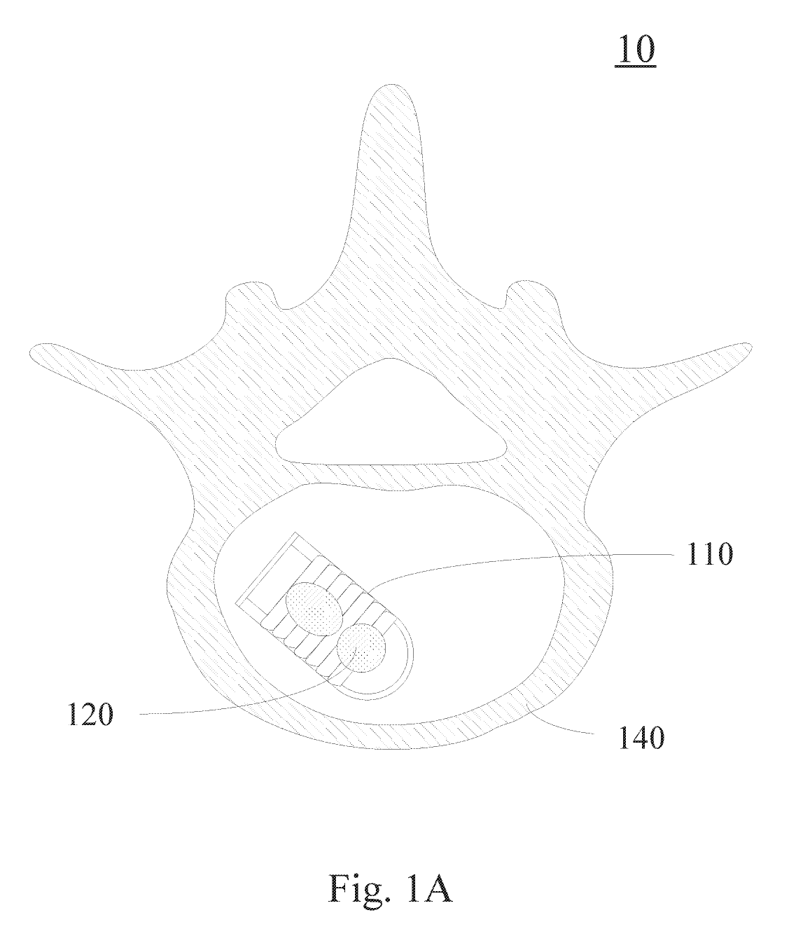

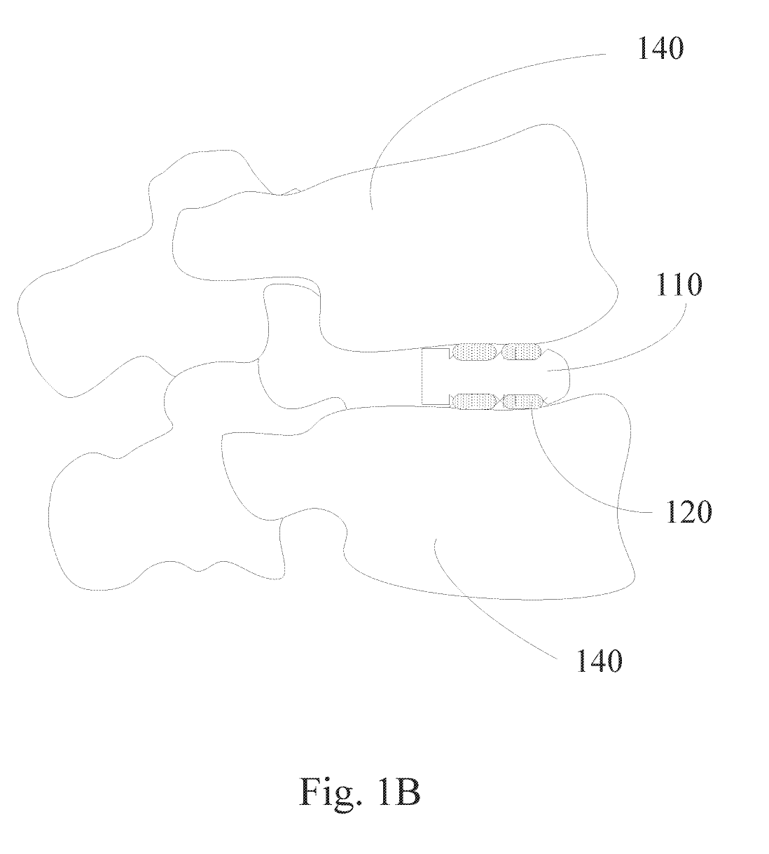

Please refer to FIGS. 1A and 1B, which show a top view and a right-side view of the intervertebral fusion apparatus for positioning and combining vertebrae in the operational state according to a preferred embodiment of the present invention. As shown in the figures, the intervertebral fusion apparatus for positioning and combining vertebrae 10 provided by the present invention is disposed between two vertebrae and comprises an intervertebral fusion cage 110 and a filling material 120. By using the contact area for connecting with top and bottom vertebrae provided by the intervertebral fusion cage 110 and the filling material 120, the friction and stability required in positioning and combining between vertebrae by the intervertebral fusion apparatus for positioning and combining vertebrae 10 can be provided.

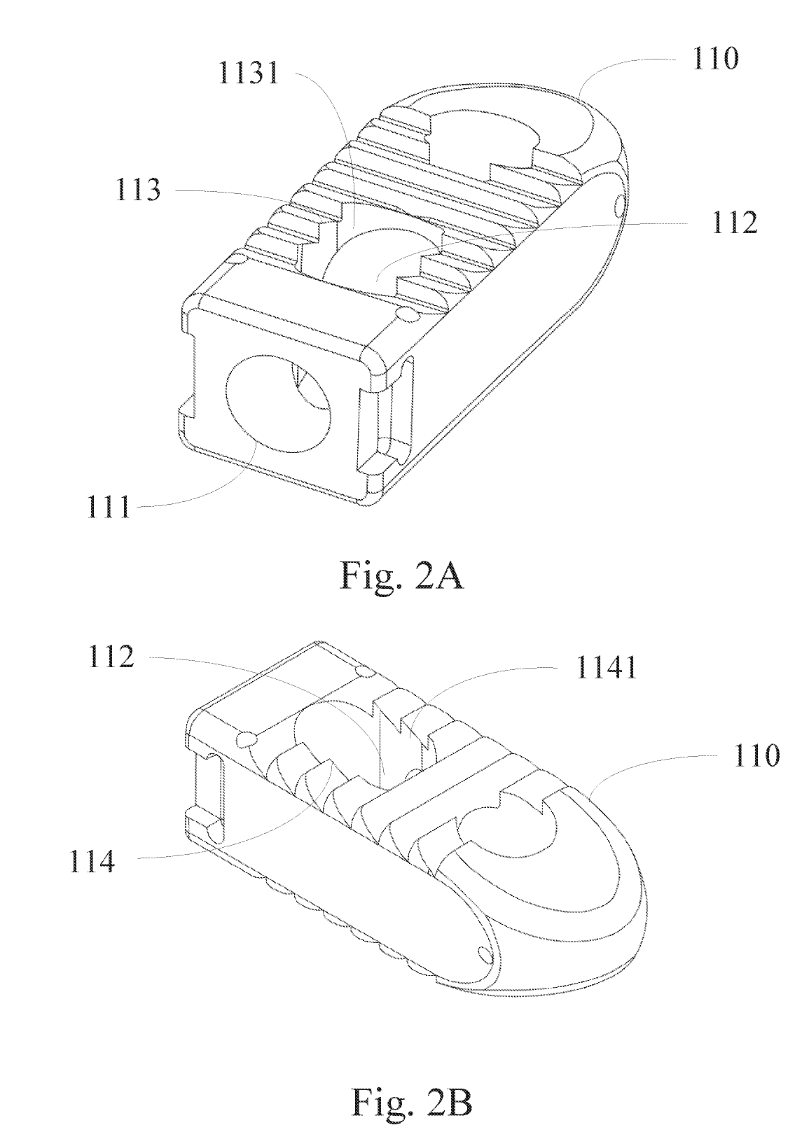

Next, please refer to FIGS. 2A and 2B, which show side views of the top and bottom surfaces of the intervertebral fusion cage of the intervertebral fusion apparatus according to a preferred embodiment of the present invention. The structure of the intervertebral fusion cage 110 of the intervertebral fusion apparatus for positioning and combining vertebrae 10 comprises an inlet 111 on the front end surface, an accommodating space 112 inside the intervertebral fusion cage 110, and one or more penetrating hole in the vertical direction. The one or more penetrating hole can be a top penetrating hole 113 or a bottom penetrating hole 114. The top penetrating hole 113 includes a top penetrating accommodating space 1131; the top penetrating hole 113 includes a bottom penetrating accommodating space 1141. Besides, the inlet 111 and the top penetrating accommodating space 1131 or the bottom penetrating accommodating space 1141 communicates with the accommodating space 112, respectively.

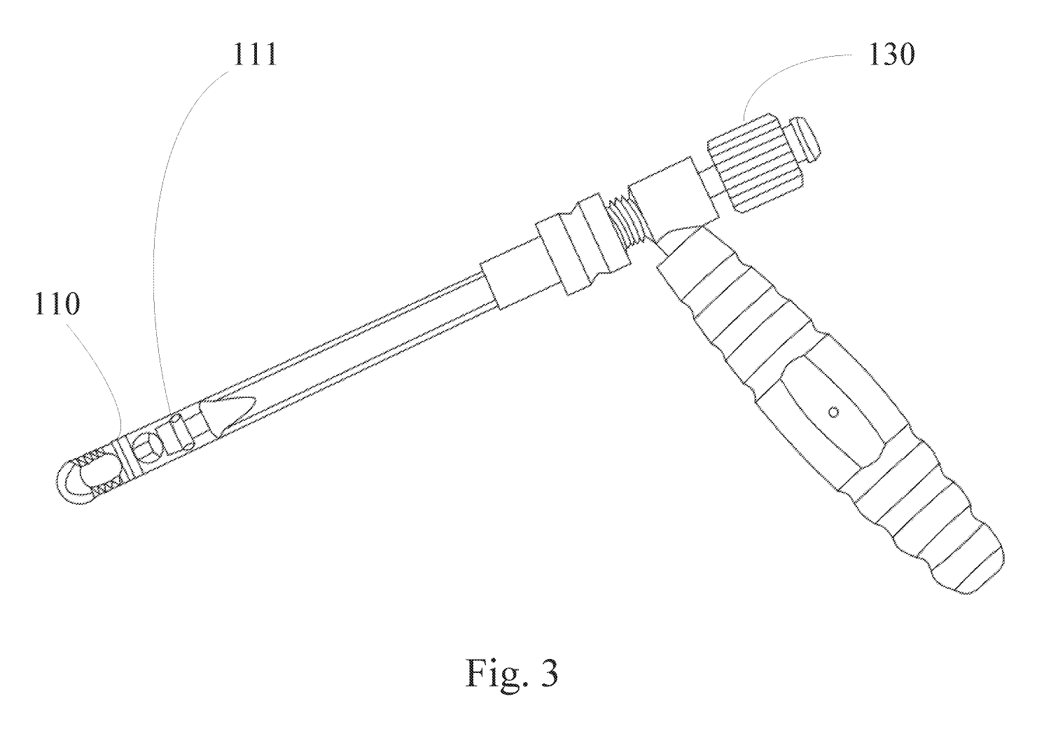

Next, please refer to FIG. 3, which shows a side view of the intervertebral fusion cage combined with the operational device according to a preferred embodiment of the present invention. As shown in the figure, according to the intervertebral fusion cage 110 provided by the present invention, the inlet 111 is not only the entrance for the filling material 120 to enter the intervertebral fusion cage 110. It can further combine with different operative apparatuses for achieving the purposes of positioning and combining vertebrae using the intervertebral fusion apparatus 10 provide by the present invention. For example, the intervertebral fusion cage 110 can combine with an implant device 130 through the inlet 111. Then the implant device 130 can deliver the intervertebral fusion cage 110 to the affected part from the outside of the patient's body. By further placing the intervertebral fusion cage 110 by striking, the purpose of delivering the intervertebral fusion cage 110 to the target region can be achieved. In addition, the intervertebral fusion cage 110 can combine with a filling-material injector 131 through the inlet 111. The filling-material injector 131 can deliver the filling material 120 to the intervertebral fusion cage 110 through the inlet 111, and fill and diffuse the filling material 120 following the filling direction as described above for achieving the purpose of delivering the filling material 120 to the affected part and positioning and combining vertebrae. Based on the above principles, the inlet 111 provided by the present invention can combine with various operative apparatuses using tight, screw, or wedge connecting methods. Furthermore, the inlet 111 provided by the present invention includes a connecting device used for connecting with the filling-material injector 131 or the implant device 130.

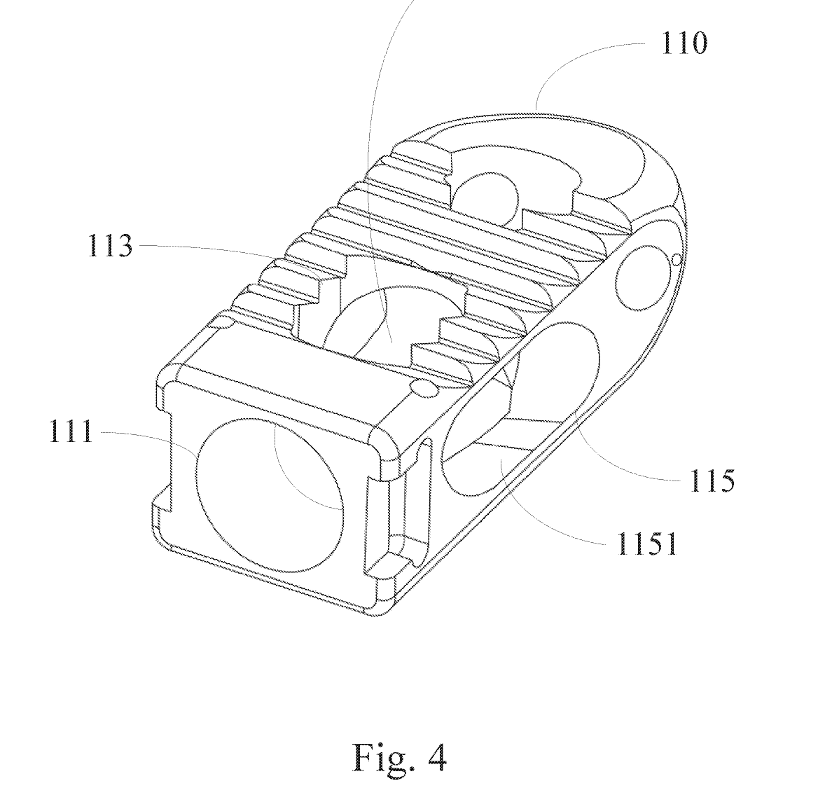

Next, please refer to FIG. 4, which shows a side view of the intervertebral fusion cage according to another preferred embodiment of the present invention. As sown in the figure, the intervertebral fusion cage 110 provided by the present invention can further include one or more side penetrating hole 115, which includes a side accommodating space 1151 communicating, likewise, with the accommodating space 112. By disposing the one or more side penetrating hole 115 and the side accommodating space 1151, the filling material 120 entering the accommodating space 112 from the inlet 111 can diffuse and flow towards the one or more side penetrating hole 115. As the filling material 120 fills to the side of the intervertebral fusion cage 110 and limits the movable range of the intervertebral fusion cage 110, the lateral width of the intervertebral fusion apparatus for positioning and combining vertebrae 10 provided by the present invention can be extended effectively, which makes the overall lateral width of the intervertebral fusion apparatus 10 is greater than the lateral width of the operation opening, for implanting the intervertebral fusion apparatus 10. Consequently, the situation of the intervertebral fusion cage slipping off the operation opening after completion of the surgery can be avoided effectively.

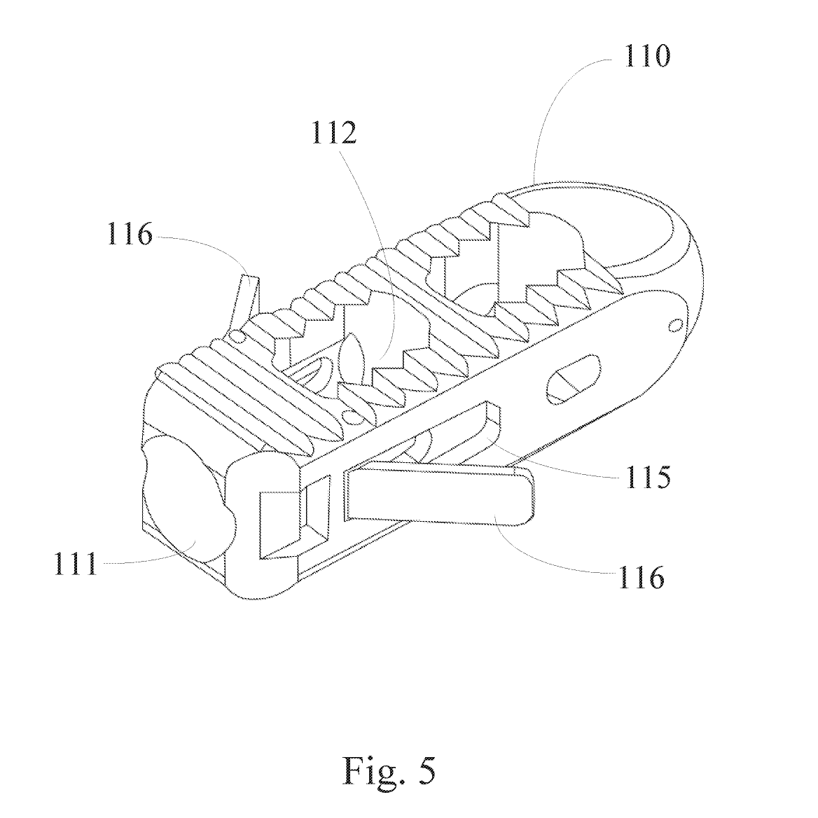

Next, please refer to FIG. 5, which shows a side view of the intervertebral fusion cage according to another preferred embodiment of the present invention. As shown in the figure, the intervertebral fusion cage 110 provided by the present invention further includes a wing member 116 pivoted on the intervertebral fusion cage 110 and disposed in the one or more side penetrating hole 115. Based on the above properties, when the filling material 120 entering the accommodating space 112 and guided by the one or more side penetrating hole 115 diffuses and flows to the side of the intervertebral cage 110, the wing member 116 will extend pivotally to the side as pushed by the filling material 120. The extended wing can increase the lateral width of the intervertebral fusion apparatus for positioning and combining vertebrae 10 provided by the present invention. In addition, the extended win can also limit the flowing direction of the filling material 120. Thereby, the wing member 116 can not only achieve the purpose of avoiding slipping off the operation opening but also control the range of limiting the intervertebral fusion cage 110 by the filling material 120 flowing in the spinal canal.

According to a preferred embodiment of the present invention, the wing member 116 pivots on the front edge of the side penetrating hole 115, namely, the front location of the intervertebral fusion cage 110, and is on the same side of the inlet 111. Based on the above design, when the wing member 116 pivots to the side and extends as pushed by the filling material 120, the extended wing can avoid effectively the spinal fusion apparatus from slipping off the operation opening. In addition, it also can prevent the filling material 120 from spilling to the other tissues of the body, which might result in unnecessary surgical danger to patients.

The filling material 120 of the intervertebral fusion apparatus for positioning and combining vertebrae 10 according to the present invention is a fluid material. The fluid material is not limited to the fluid form. Any material that is distributive and able to flow to the accommodating space 112, the top accommodating space 1131, the bottom accommodating space 1141, and the side accommodating space 1151 can be the filling material 120 according to the present invention. Based on the above description, the filling material 120 according to the present invention can be filling particles, a solidifiable material, or a thermoplastic fluid.

The filling particles provided by the present invention are highly fluid and distributive for being distributed into the intervertebral fusion cage and the spinal canal. By filling completely the space inside the intervertebral fusion cage and the spinal canal, the intervertebral fusion apparatus for positioning and combining vertebrae is left with no space to move and hence fixed inside the spinal canal. Based on the above properties, the filling particles provided by the present invention can be ceramic particles.

The solidifiable material provided by the present invention is fluid. It is a material starting the solidification reaction by time or temperature changes. Based on the above principle, the solidifiable material provided by the present invention is in a fluid form. The fluid can be, but not limited to, plastics or polymer solution in the liquid, gel, or melt state. Besides, in order to achieve the purpose of starting the solidification reaction by time or temperature changes as described above, the fluid provided by the present invention is preferably thermoplastic fluid or fluid doped with curing agent. Based on the above principle, the filling material 120 is selected from the group consisting of amorphous calcium phosphate, poorly crystalline calcium phosphate, hydroxyapatite, carbonated apatite (calcium-deficient hydroxyapatite), monocalcium phosphate, calcium metaphosphate, heptacalcium phosphate, dicalcium phosphate dihydrate, tetracalcium phosphate, octacalcium phosphate, calcium pyrophosphate, tricalcium phosphate, calcium sulfate hemihydrate, calcium sulfate dihydrate, polysaccharides, nucleic acids, carbohydrates, proteins, polypeptides, poly(α-hydroxy acids), poly(lactones), poly(amino acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide and copolymers thereof, alginic acid, arabic gum, guar gum, xantham gum, gelatin, chitin, chitosan, chitosan acetate, chitosan lactate, chondroitin sulfate, N,O-carboxymethyl chitosan, dextran (e.g., α-cyclodextrin, β-cyclodextrin, γ-cyclodextrin, or sodium dextran sulfate), fibrin glue, glycerol, hyaluronic acid, sodium hyaluronate, cellulose (e.g., methylcellulose, carboxy methylcellulose, hydroxypropyl methylcellulose, or hydroxyethyl cellulose), glucosamine, proteoglycan, starch (e.g., hydroxyethyl starch or starch soluble), lactic acid, pluronic, sodium glycerophosphate, collagen, glycogen, keratin, silk fibroin and the combination thereof. Preferably, the solidifiable material provided by the present invention is a bioabsorbable material or a biodegradable material.

In the following, please refer to FIGS. 1A, 1B, and 2. They are used for describing the relations among the components of the intervertebral fusion apparatus for positioning and combining vertebrae 10 provided by the present invention. As shown in the figures, according to the intervertebral fusion apparatus for positioning and combining vertebrae 10 provided by the present invention, the filling material 120 is injected from the inlet 111 located at the front of the intervertebral fusion cage 110. Afterwards, the filling material 120 enters the accommodating space 112 communicating with the inlet 111 and starts filling. Meanwhile, during the process of the filling material 120 filling the accommodating space 112 or after the accommodating space 112 is filled full, a part of the filling material 120 will flow and diffuse to the top or bottom accommodating space 1131, 1141 communicating with the accommodating space 112, and then spill out of the intervertebral fusion cage 110 from the top or bottom penetrating hole 113, 114. At this time, the spilled filling material 120 will contact a vertebra 140 for increasing the contact area between the intervertebral fusion cage 110 and the vertebra 140. Besides, when the spilled filling material 120 is increased, the filling material 120 will extend to the front end and rear end of the intervertebral fusion cage 110 along a top surface 117 and a bottom surface 118 for providing more contact surface area. By means of the above process, the intervertebral fusion apparatus provided by the present invention can improve the stability and thus giving the intervertebral fusion apparatus for positioning and combining vertebrae 10 according to the first embodiment of the present invention.

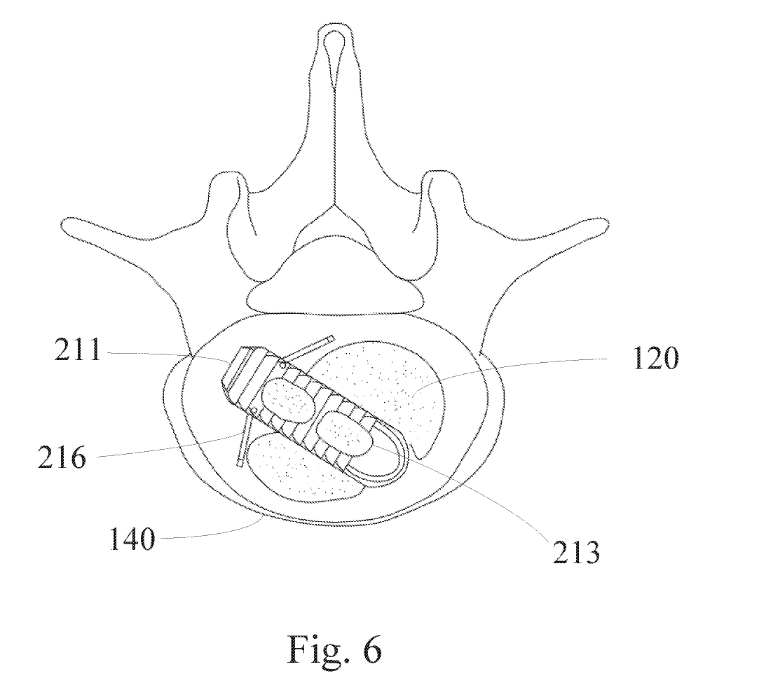

Next, please refer to FIG. 5 and FIG. 6. FIG. 6 shows a top view of the intervertebral fusion apparatus for positioning and combining vertebrae in the operational state according to another preferred embodiment of the present invention. As shown in the figures, the present provides an intervertebral fusion apparatus 20 in another form disposed between two vertebrae. The structure of the intervertebral fusion apparatus 20 comprises an intervertebral fusion cage 210 and one or more wing member 216 pivoted on the intervertebral fusion cage 210. The intervertebral fusion cage 20 includes an inlet 211 on the front end surface and an accommodating space 212 therein. The inlet 211 communicates with the accommodating space 212. The intervertebral fusion cage 210 includes one or more side penetrating hole 215 in the lateral direction and communicating with the accommodating space 212. In addition, the one or more wing member 216 pivots on the intervertebral fusion cage 20 and is disposed in the one or more side penetrating hole 215, such that the one or more wing member 216 can extend outwards, facilitating contacting and connecting effects. Thereby, when the intervertebral fusion apparatus 20 is positioned, an external force or an inertial force can be exerted on the intervertebral fusion apparatus 20 to deform the one or more wing member 216. The deformed one or more wing member 216 will contact the sidewall of the vertebra 140 or be against the sidewall of the vertebra 140 for producing friction or resistance and thus achieving the purpose of stabilizing the intervertebral fusion apparatus 20 inside vertebrae. Based on the above principle, the one or more wing member 216 provided by the present invention is not limited to pivoting on the fronted or rear end of the intervertebral fusion cage 210. That is to say, the one or more wing member 216 is not limited to pivoting on the front edge or rear edge of the one ore more side penetrating hole 215.

According to the intervertebral fusion apparatus 20 as described above, the intervertebral fusion cage 210 can further include atop penetrating hole 213 and a bottom penetrating hole 214 communicating with the accommodating space 212. The top and bottom holes 213, 214 include a top accommodating space 2131 and a bottom accommodating space 2141, respectively, and are operated along with the filling material 120, such that the wing member 216 can be pushed and pivoted by the filling material 120 and thus extending to the side of the intervertebral fusion cage 210. The relative positions of components, the operational direction of components, and the achieved effects of the intervertebral fusion apparatus 20 operating concurrently with the filling material 120 are identical to those of the intervertebral fusion apparatus 10. Hence, the details will not be described again.

To sum up, the present invention indeed provides an intervertebral fusion apparatus for positioning and combining vertebrae. It can overcome the shortcomings of instability during implanting and slipping off intervertebral disc easily after implantation due to insufficient contact area between the intervertebral fusion cage and vertebrae. According to the present invention, by using a solidifiable material and the top and bottom penetrating holes in the intervertebral fusion cage, the solidifiable material can be added continuously for contacting the top and bottom vertebrae and hence increasing the contact area friction, and adhesion between the overall structure and the vertebrae. Consequently, the required stability during the early stage of a surgery for the intervertebral fusion apparatus for positioning and combining vertebrae can be improved effectively. In addition, by using the design of the side penetrating hole and the wing member, the overall lateral width of the intervertebral fusion apparatus is increased and thus avoiding slipping off the surgery site after completion of surgery. The design can further control the filling direction of the solidifiable material as used concurrently with the solidifiable material and prevent the solidifiable from contacting the human tissues other than the surgery site, which might lead to unnecessary harms. Thereby, the present invention indeed provides an intervertebral fusion apparatus for positioning and combining vertebrae having high operational stability and safely and improves the technical standard of the related field.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

What is claimed is:

1. An intervertebral fusion apparatus for positioning and combining vertebrae, disposed between two vertebrae, and comprising:
an intervertebral fusion cage, including an inlet on the front surface, an accommodating space, and a top penetrating hole and a bottom penetrating hole in the vertical direction, said inlet, said top penetrating hole, and said bottom penetrating hole communicating with said accommodating space, and said top penetrating hole and said bottom penetrating hole including a top accommodating space and a bottom accommodating space, respectively; and
a filling material, injecting from said inlet, entering said accommodating space, and flowing to said top accommodating and said bottom accommodating space.

2. The intervertebral fusion apparatus for positioning and combining vertebrae of claim 1, wherein said inlet of said intervertebral fusion cage includes a connecting device used for connecting with an injector.

3. The intervertebral fusion apparatus for positioning and combining vertebrae of claim 1, wherein said filling material enters and fills said top accommodating space or said bottom accommodating space through said accommodating space, and said filling material entering and filling said top accommodating space or said bottom accommodating space will contact a top vertebra or a bottom vertebra.

4. The intervertebral fusion apparatus for positioning and combining vertebrae of claim 1, wherein said intervertebral fusion cage includes one or more side hole having a side accommodating space communicating with said accommodating space.

5. The intervertebral fusion apparatus for positioning and combining vertebrae of claim 4, wherein said intervertebral fusion cage includes a wing member pivoted on said intervertebral fusion cage and located outside of said one or more side hole.

6. The intervertebral fusion apparatus for positioning and combining vertebrae of claim 5, wherein said wing member is pushed by said filling material injected into said accommodating space and extending pivotally to the side.

7. The intervertebral fusion apparatus for positioning and combining vertebrae of claim 5, wherein said wing member is pivoted on the front edge of said intervertebral fusion cage.

8. The intervertebral fusion apparatus for positioning and combining vertebrae of claim 1, wherein said filling material includes filling particles, a solidifiable material, or a thermoplastic fluid.

9. The intervertebral fusion apparatus for positioning and combining vertebrae of claim 1, wherein said solidifiable material is selected from the group consisting of amorphous calcium phosphate, poorly crystalline calcium phosphate, hydroxyapatite, carbonated apatite (calcium-deficient hydroxyapatite), monocalcium phosphate, calcium metaphosphate, heptacalcium phosphate, dicalcium phosphate dihydrate, tetracalcium phosphate, octacalcium phosphate, calcium pyrophosphate, tricalcium phosphate, calcium sulfate hemihydrate, calcium sulfate dihydrate, polysaccharides, nucleic acids, carbohydrates, proteins, polypeptides, poly(α-hydroxy acids), poly(lactones), poly(amino acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide, alginic acid, arabic gum, guar gum, xantham gum, gelatin, chitin, chitosan, chitosan acetate, chitosan lactate, chondroitin sulfate, N,O-carboxymethyl chitosan, dextran (e.g., α-cyclodextrin, β-cyclodextrin, γ-cyclodextrin, or sodium dextran sulfate), fibrin glue, glycerol, hyaluronic acid, sodium hyaluronate, cellulose e.g., methylcellulose, carboxy methylcellulose, hydroxypropyl methylcellulose, or hydroxyethyl cellulose), glucosamine, proteoglycan, starch (e.g., hydroxyethyl starch or starch soluble), lactic acid, pluronic, sodium glycerophosphate, collagen, glycogen, keratin, silk fibroin and the combination thereof.

10. An intervertebral fusion apparatus for positioning and combining vertebrae, disposed between two vertebrae, and comprising:
an intervertebral fusion cage, including an inlet on the front surface and an accommodating space, said inlet communicating with said accommodating space, including one ore more side hole in the lateral direction, said one ore more side hole including a side accommodating space communicating with said accommodating space; and
one or more wing member, pivoted on said intervertebral fusion cage and located outside said side hole, and outward extendable for contacting and connecting.

11. The intervertebral fusion apparatus for positioning and combining vertebrae of claim 10, wherein said wing members are pivoted on the front edge of said intervertebral fusion cage and opposing to one another.

12. The intervertebral fusion apparatus for positioning and combining vertebrae of claim 10, wherein said inlet of said intervertebral fusion cage includes a connecting device used for connecting with an injector.

13. The intervertebral fusion apparatus for positioning and combining vertebrae of claim 10, wherein said intervertebral fusion cage includes a top penetrating hole and a bottom penetrating hole; said top penetrating hole and said bottom penetrating hole include a top accommodating space and a bottom accommodating space, respectively; and said top accommodating space and said bottom accommodating space communicate with said accommodating space.

14. The intervertebral fusion apparatus for positioning and combining vertebrae of claim 13, wherein said accommodating space includes a filling material flowing to said top accommodating space, said bottom accommodating space, and said side accommodating space for filling.

15. The intervertebral fusion apparatus for positioning and combining vertebrae of claim 14, wherein said filling material includes filling particles, a solidifiable material, or a thermoplastic fluid.

16. The intervertebral fusion apparatus for positioning and combining vertebrae of claim 15, wherein said solidifiable material is selected from the group consisting of amorphous calcium phosphate, poorly crystalline calcium phosphate, hydroxyapatite, carbonated apatite (calcium-deficient hydroxyapatite), monocalcium phosphate, calcium metaphosphate, heptacalcium phosphate, dicalcium phosphate dihydrate, tetracalcium phosphate, octacalcium phosphate, calcium pyrophosphate, tricalcium phosphate, calcium sulfate hemihydrate, calcium sulfate dihydrate, polysaccharides, nucleic acids, carbohydrates, proteins, polypeptides, poly(α-hydroxy acids), poly(lactones), poly(amino acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide, alginic acid, arabic gum, guar gum, xantham gum, gelatin, chitin, chitosan, chitosan acetate, chitosan lactate, chondroitin sulfate, N,O-carboxymethyl chitosan, dextran (e.g., α-cyclodextrin, β-cyclodextrin, γ-cyclodextrin, or sodium dextran sulfate), fibrin glue, glycerol, hyaluronic acid, sodium hyaluronate, cellulose (e.g., methylcellulose, carboxy methylcellulose, hydroxypropyl methylcellulose, or hydroxyethyl cellulose), glucosamine, proteoglycan, starch (e.g., hydroxyethyl starch or starch soluble), lactic acid, pluronic, sodium glycerophosphate, collagen, glycogen, keratin, silk fibroin and the combination thereof.

17. The intervertebral fusion apparatus for positioning and combining vertebrae of claim 14, wherein said wing member is pushed by said filling material injected into said accommodating space and flowing to said side accommodating space for filling and extends pivotally to the side.

18. The intervertebral fusion apparatus for positioning and combining vertebrae of claim 17, wherein said filling material includes filling particles, a solidifiable material, or a thermoplastic fluid.

19. The intervertebral fusion apparatus for positioning and combining vertebrae of claim 18, wherein said solidifiable material is selected from the group consisting of amorphous calcium phosphate, poorly crystalline calcium phosphate, hydroxyapatite, carbonated apatite (calcium-deficient hydroxyapatite), monocalcium phosphate, calcium metaphosphate, heptacalcium phosphate, dicalcium phosphate dihydrate, tetracalcium phosphate, octacalcium phosphate, calcium pyrophosphate, tricalcium phosphate, calcium sulfate hemihydrate, calcium sulfate dihydrate, polysaccharides, nucleic acids, carbohydrates, proteins, polypeptides, poly(α-hydroxy acids), poly(lactones), poly acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxy butyrate poly(ε-caprolactone), poly(δ-valerolactone), poly(γ-butyrolactone), poly(caprolactone), polyacrylic acid, poly carboxylic acid, poly(allylamine hydrochloride), poly(diallyldimethylammonium chloride), poly(ethyleneimine polypropylene fumarate, polyvinyl alcohol, polyvinylpyrrolidone, polyethylene, polymethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide, alginic acid, arabic gum, guar gum, xantham gum, gelatin, chitin, chitosan, chitosan acetate, chitosan lactate, chondroitin sulfate, N,O-carboxymethyl chitosan, dextran (e.g., α-cyclodextrin, β-cyclodextrin, γ-cyclodextrin, or sodium dextran sulfate), fibrin glue, glycerol, hyaluronic acid, sodium hyaluronate, cellulose (e.g., methylcellulose, carboxy methylcellulose, hydroxypropyl methylcellulose, or hydroxyethyl cellulose), glucosamine, proteoglycan, starch (e.g., hydroxyethyl starch or starch soluble), lactic acid, pluronic, sodium glycerophosphate, collagen, glycogen, keratin, silk fibroin and the combination thereof.
